(19) United States
(12) Patent Application Publication
(10) Pub. No.: US 2009/0199608 A1
(43) Pub. Date: Aug. 13, 2009

(54) COMPOSTING APPARATUS, INSTALLATION AND METHOD THEREOF

(75) Inventors: Carl Genois, Quebec (CA); Rolf Arneberg, Quebec (CA); Denis Potvin, Quebec (CA); Bernad Caron, Montreal (CA); Jose Scorsone, Quebec (CA)

Correspondence Address: GOUDREAU GAGE DUBUC, 2000 MCGILL COLLEGE, SUITE 2200, MONTREAL, QC, H3A 3H3, CA

(73) Assignee: CONPOREC INC, Quebec, QC (CA)

(21) Appl. No.: 12/281,252

(22) PCT Filed: Mar. 2, 2007
(86) PCT No.: PCT/CA2007/000351

§ 371 Date: Jan. 7, 2009

Related U.S. Application Data
(60) Provisional application No. 60/778,087, filed on Mar. 2, 2006.

(51) Int. Cl. C05F 11/08; C12M 1/04

(52) U.S. Cl. 71/9; 435/290.4; 71/14

(57) ABSTRACT

There is disclosed a system and method for composting large amounts waste material comprising a predominant amount of compostable organic material, such as Source Separated Organic (SSO) material. The method comprises mixing the waste material with a bulking agent such as tree bark, removing larger inorganic material then compositing the remainder using compositing bacteria and forced air to promote composting. During its initial composting stages when the majority of noxious gasses, fumes and odours are generated the composting is carried out in an enclosed area and the gases removed and treated using a bio-filter. Once the compostable organic material has been adequately decomposed the bulking agent is removed along with any residual inorganic material and the composting process completed. In particular embodiment the bulking agent is recycled. There is also disclosed a method and device for mixing the waste material with the bulking agent.

### FIELD OF THE INVENTION

The present invention relates to composting apparatus, installation and method. More specifically, the present invention is concerned with organic composting.

### BACKGROUND OF THE INVENTION

Municipalities and cities desire to reduce waste. Paper, steel, glass and plastic are already recycled. Organic materials such as fruits, vegetables and other leftovers are not recycled on the same basis. However, organic material can be recycled into compost. Recycling large amount of organic materials requires an industrial sized processing and composting plan. Additionally, manipulating, processing and composting organic material generate undesirable odours. Wastewater is also generated by organic material processing and composting.

Manipulating and processing organic material in order to obtain good quality compost is a significant task, which is performed over an extended period of time. The layout and disposition of the infrastructures required for performing each step in the process must be carefully analysed to limit movement of the composting material to that, which is necessary.

### SUMMARY OF THE INVENTION

In order to address the above and other drawbacks there is provided a system for treating a waste material comprising a predominant amount by weight of a compostable organic material. The system comprises a mixer for combining a bulking agent with the waste material, a first sorting device for removing predominantly inorganic material having a dimension exceeding a predetermined amount from the bulked waste material, a first phase compositing area located in an enclosed area for receiving the sorted waste material on a floor thereof, the first phase area further comprising a first network of drains and a first network of air ducts imbedded in the first phase compositing area floor, the first network of drains leading to a cistern and the first network of air ducts in operative engagement with a blower for blowing air out of the first network of air ducts, a means for increasing a humidity of the sorted waste material, a first network of vents for collecting noxious gases emitted by the composting process when compositing the screened waste material and transmitting the emitted gases to a bio-filter, a second sorting device for removing residual organic material and the bulking agent from the partially composted sorted waste material, a transition area for receiving partially composted screened waste material from the first phase compositing area on a floor thereof, a second phase compositing area for receiving the compostable organic material from the transition area on a floor thereof, the second phase area comprising a second network of drains and a second network of air ducts imbedded in the second phase compositing area floor, the second network of drains leading to the cistern and the second network of air ducts in operative engagement with the blower for blowing air through the second network of air ducts and a means for increasing a humidity of the compostable organic material, and a curing phase compositing area for storing composted organic material.

There is also disclosed a method for treating a waste material comprising a predominant amount by weight of a compostable organic material. The method comprises during a conditioning phase adding a bulking agent to the waste material, pretreating the bulked waste material to remove predominantly inorganic material having at least one dimension of greater than a predetermined amount, during an initial composting phase piling the pretreated waste material into a first heap, inoculating the heap with a composting bacteria, promoting drying and compositing of the pretreated waste material in the heap by permeating air into the heap, collecting gases generated by the composting bacteria during compositing the pretreated waste material, and treating the collected gases in a bio-filter, during a subsequent composting phase removing residual inorganic material and a majority of the bulking agent from the dried and partially composted waste material to yield partially composted organic material, readjusting a humidity of the partially composted organic material by adding water, and piling the partially composted organic material into a second heap, and promoting drying and compositing of the partially composted organic material by permeating air into the heap, during a curing (or maturing) phase: removing residual bulking agent from the dried composted organic material, and storing the dried composted organic material.

Furthermore, there is disclosed a method for compositing waste material comprising a predominant amount of compostable organic material. The method comprises homogenously mixing the waste material with a bulking agent, wherein a predominant amount of the bulking agent is recycled bulking agent, spraying the waste material and bulking agent mix with a water containing a composting bacteria, promoting drying and partial compositing of the composting waste material by permeating air through the waste material and bulking agent mix, separating the bulking agent from the partially composted waste material to yield partially composted organic material and the recycled bulking agent, spraying the partially composted waste material with a water containing a composting bacteria, and promoting drying and compositing of the compostable organic material by permeating air through the waste material and the bulking agent.

Also, there is disclosed a method for mixing waste material comprising a predominant amount of compostable organic material with a bulking agent. The method comprises providing a mixer comprising a mixing tub comprising an inverted frusto-conical shape and a closed lower end and a vertical auger mounted for rotation in the lower end, the auger comprised of at least one exposed helical flighting extending from a frusto-conical hub, placing the waste material in the mixing tub, mixing the waste material by rotating the auger in a direction of the helical flighting at a first speed, adding the bulking agent to the mixed waste material, and mixing the bulking agent and the waste material by rotating the auger in a direction of the helical flighting at a second speed greater than the first speed.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 is a schematic diagram of the plant layout for manipulating, processing and composting organic material, in accordance with an illustrative embodiment of the present invention;

FIG. 2 is a schematic diagram of the reception and pre-treatment area, in accordance with an illustrative embodiment of the present invention;

FIG. 3 is a schematic diagram of mixer, in accordance with an illustrative embodiment of the present invention;

FIG. 4 is a schematic diagram of the first composting phase area and transition area, in accordance with an illustrative embodiment of the present invention;

FIG. 5 is a schematic diagram of the second composting phase area, in accordance with an illustrative embodiment of the present invention;

FIG. 6 is a schematic diagram of the curing and storage area, in accordance with an illustrative embodiment of the present invention; and

FIG. 7 is a schematic diagram of the biofilter, in accordance with an illustrative embodiment of the present invention.

### DETAILED DESCRIPTION OF THE ILLUSTRATIVE EMBODIMENTS

The present invention is illustrated in further details by the following non-limiting examples.

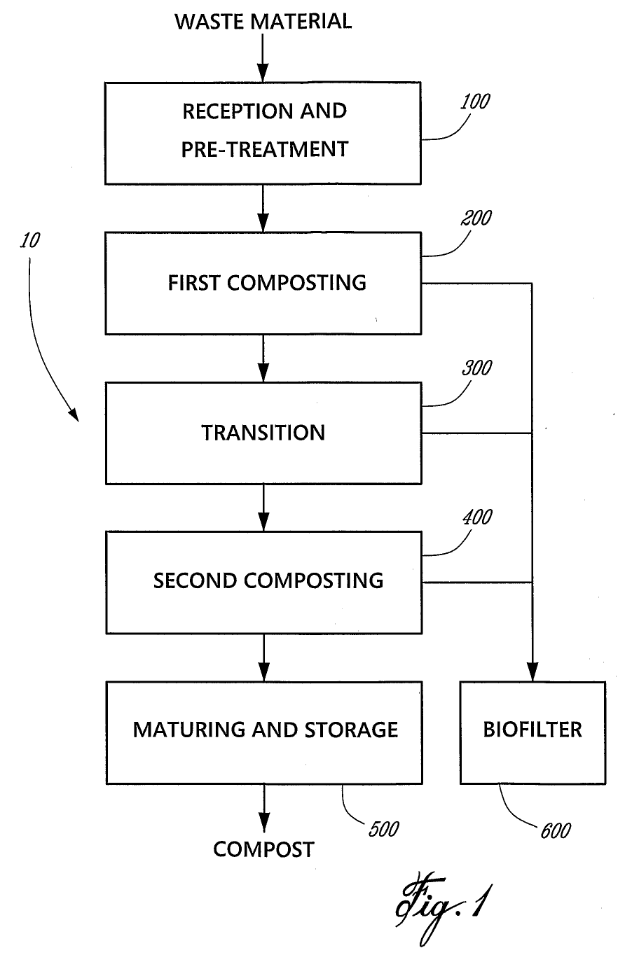

Referring to FIG. 1, and in accordance with an illustrative embodiment of the present invention, a system for composting organic matter, generally referred to using the reference numeral 10, will now be described. The system 10 comprises of a reception and pre-treatment area 100, a first composting phase area 200, a transition area 300, a second composting phase area 400, and a curing and storage area 500. Combining these areas, which each corresponds to a different phase of the composting process, also referred to as a Source Separated Organic (SSO) process, the system 10 is adapted to receive and further treat compostable organic material provided by collection of waste material to produce a finished compost product, which will ultimately be routed to the compost market.

Throughout the composting process, the system 10 segregates organic and inorganic materials by simple mechanical procedures while confining and/or treating liquid and gaseous emissions. A sequence of drying the organic matter, segregating organic from inorganic matters and re-humidification between composting phases is implemented to improve the end compost product while at the same time reducing the cycle time. As the material to be composted may come from domestic collection and thus contain a significant quantity of undesirable inorganic residues, the waste material is dried to allow efficient separation of organic and inorganic material by mechanical equipment. This is done using forced aeration as the main composting method, thus allowing evaporation of the large quantities of water contained in the organic material. In addition, all compost activities likely to release noxious fumes are confined within a building having a sealed reinforced concrete surface protected by a roof to significantly reduce the risk of diffusion of the noxious fumes into the surrounding environment. For this purpose, the reception and pre-treatment area 100, the first composting phase area 200, the transition area 300, as well as the second composting phase area 400 are located in this closed building, which is under negative pressure on a permanent basis. Noxious emissions extracted throughout the plant are further treated using a biofilter 600. As a result, significant amounts of organic material can be composted without generating undesirable odours.

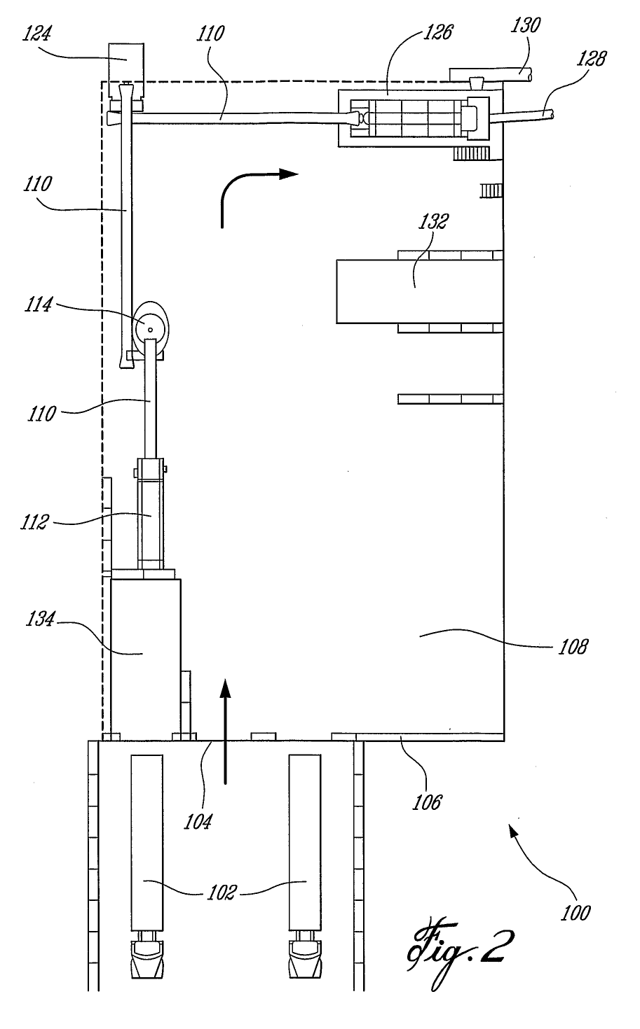

Referring now to FIG. 2 in addition to FIG. 1, the reception and pre-treatment area 100 will now be described. Trucks 102 bringing humid waste material to the plant are illustratively unloaded under a confined atmosphere at one of three access doors 104 of a dock 106 and the unloaded waste material is stored in a storage area 108. The reception and pre-treatment area 100, which illustratively has a capacity of about 500 cubic meters on a sealed reinforced concrete floor surface of about 500 square meters, is kept under negative pressure using a ventilation system (not shown). The negative pressure is further maintained by ensuring that two doors of the building are not opened at the same time. As seen on FIG. 1, the reception and pre-treatment area 100 further allows for excess wastewater discharged from the unloaded waste material to be collected and drained to a cistern (not shown) for later reuse in the composting process. It is desirable for waste material received at the reception and pre-treatment area 100 to be treated and sent to the first composting phase area 200 as soon as possible, illustratively within 12 hours following arrival at the earliest and within 72 hours at the latest, in order to take into account possible contingencies, process halts due maintenance, as well as to facilitate the management of supplies.

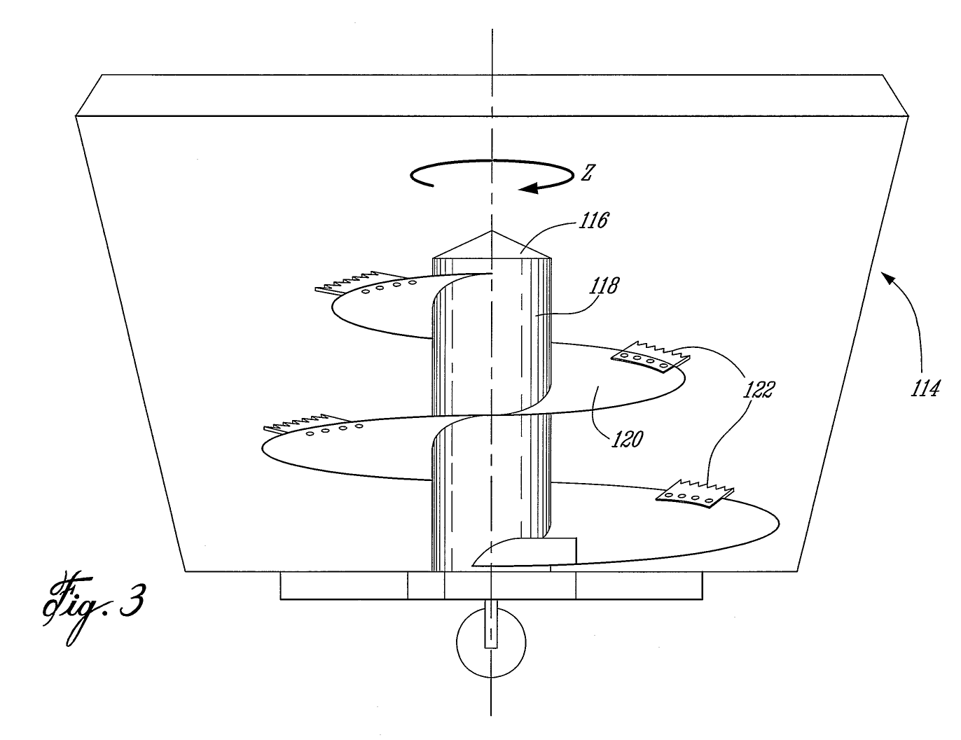

Referring now to FIG. 3 in addition to FIG. 2, waste material from the storage area 108 is routed to a conveyor belt 110 via a feed hopper 112. From the conveyor belt 110, the waste material is then fed to a mixer 114 comprising a mixing shaft 116 of generally inverted frusto conical or cylindrical shape and a closed lower end vertical auger 118 mounted for rotation. As seen in FIG. 3, the auger 118 is used to move the waste material by means of a rotating helical flighting 120 about an axis of rotation Z. Serrated blades 122 are further mounted on an outer edge of the helical flighting 120. The mixer 114 initially operates at a relatively high speed (illustratively between 100 and 200 revolutions per minute (rpm)). As the waste material is contained predominantly in plastic bags, rotation of the auger 118 at this relatively high speed allows the serrated blades 122 to tear the bags thereby liberating the waste material contained therein. On the other hand, the speed of rotation should not be excessive, as this would result in the plastic bags being shredded into fine pieces which are much more difficult to remove during subsequent sorting steps. The “bag-removal process” thus liberates the waste material which is predominantly collected in plastic bags thereby providing for increased porosity and thus facilitating air flow through the waste material at a subsequent stage. During this “bag-removal process”, the serrated blades 122 of helical flighting 120 rip open the plastic bags, thus liberating the organic material contained therein. Once the plastic bags have been opened, the speed of the mixer 114 is decreased (illustratively to between 50 and 80 rpm) to proceed with mixing of the organic material with a bulking agent. The use of a bulking agent, typically consisting of waste from the forestry industry (e.g. fresh or recycled bark, chips or peels), ensures proper separation of the composted organic material and improves the material's permeability, thus further promoting airflow and development of composting bacteria during subsequent composting stages.

The mix obtained is rapidly emptied and fed to a first sorting device, for example a trommel (or cylindrical screen) 126 or the like via other conveyor belts 110 and a buffer feed hopper 124, which reduces the amount of material rejected by ensuring a gradual and constant feed of the organic material from the mixer 114 to the trommel 126. As known in the art, such trommels comprise a cylindrical wall having a plurality of holes therein. As the trommel 126 is rotated material which is smaller than the ID of the holes escapes the trommel 126 under force of gravity. Typically, the ID of the holes decreases gradually along the length of the trommel. According to the desired throughput from the device, the trommel 126 may be raised by a few degrees, illustratively from about 0 to 10 degrees, from the horizontal. Adjusting the angle of inclination of the trommel 126 will affect the amount of organic material that is eliminated. Typically, the trommel 126 separates the organic material from components of large size, illustratively between 2 and 3 inches. Once the organic material has been pre-treated as described herein above, it is ready to be composted and is routed via a conveyor belt 128 to the first composting phase area 200 while the pre-treatment residues (large undesirable material) are routed via another conveyor belt 130 for burial.

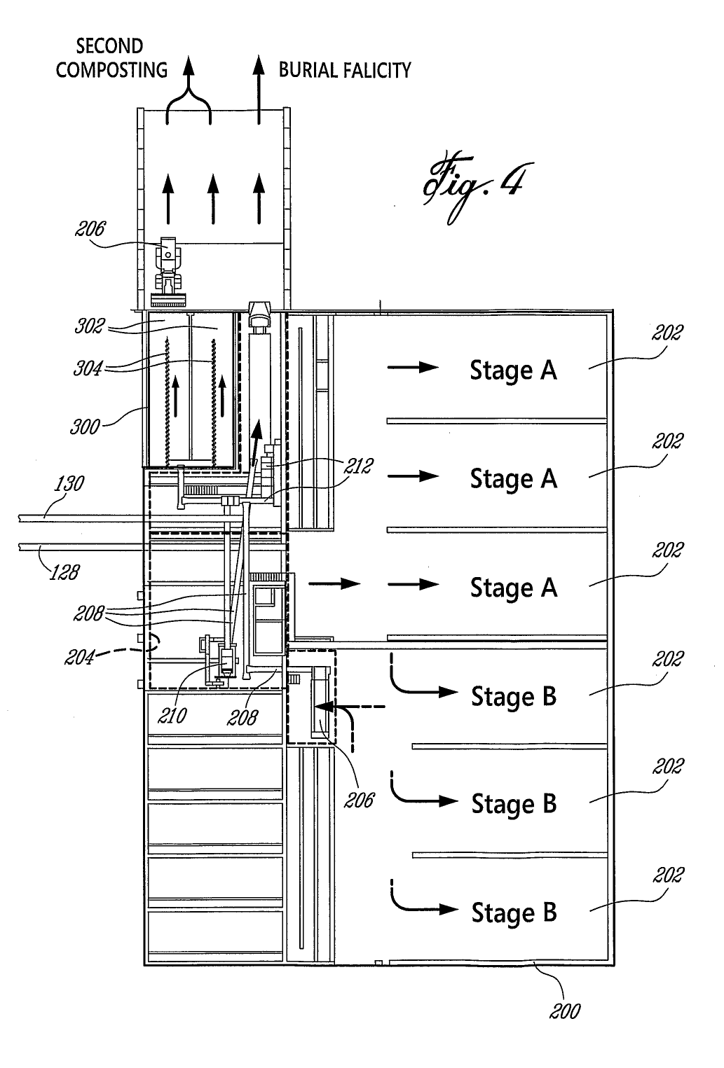

Referring now to FIG. 4, as the pre-treated material reaches the first composting phase area 200 via conveyor belt 128, it is piled into a heap in one of three modules, each module comprising two cells 202 corresponding to two stages A and B of the composting process, for a total of six juxtaposed cells 202. The cells 202 are illustratively constructed using impervious concrete and have different capacities. Indeed, due to the reduction in the volume of the compost material between stages A and B, stage A cells have a higher capacity (e.g. 750 cubic meters) than stage B cells (e.g. 675 cubic meters). The cells 202 are illustratively defined by three reinforced concrete walls and are insulated against thermal losses. A network of air ducts (not shown) is further embedded within the concrete floor of each cell 202 in order to diffuse air under the heaps of organic material in a uniform manner, thus promoting drying and composting of the material. The ducts, which constitute an air distribution network, further constitute drains which enable capture and drainage of water used throughout the composting process towards an impervious cistern (not shown) made of prefabricated concrete. It is desirable for the air distribution system to be resilient enough to withstand movement of heavy loading (comprising, for example, front loading tractors 206 and the like) equipment thereon. Composting stages A and B illustratively each have a retention time of two weeks, with mixing and re-humidifying processes being required when the organic material moves from stage A to stage B. These processes comprise spraying a top layer of organic material from a stage A cell with water, homogeneously mixing it and re-piling the wet mixed organic material in a stage B cell. A loading device, such as the loader on wheels 206, can be used for mixing and moving the organic material between the stages, with the nominal load height of each cell 202 being of about 2.5 meters.

In addition, a strategy prescribed by the Process to further Reduce Pathogens (PFRP) is adopted to prevent contamination, that is temperatures within a heap are illustratively maintained above 55 degrees Celsius for three consecutive days in the cells 202 of the first composting stage 200. As the temperatures produced during the composting process combined with their duration serves to kill many pathogens which are typically in waste material, in order to reduce contamination of partially composted waste material during its transfer from the stage A cells 202 to the stage B cells 202, a different loader as in 206 is used for initially filling the stage A cells 202 with fresh uncomposted waste material from the loader that is used to move and mix the partially composted material from the stage A cells 202 to the stage B cells 202. Alternatively, the same effect can be achieved by exchanging the bucket used on the loader, etc.

Still referring to FIG. 4, each cell 202 is illustratively equipped with a dedicated blower (not shown), which is used to provide adequate and cyclical air circulation within the cell 202 by blowing air out of the air distribution ducts. The blowers are controlled by a Programmable Logic Controller (PLC) or the like, which provides for cyclic permeation of the heaps to be adjusted to maintain the organic material at a pre-determined temperature (illustratively at least 55° C.). Illustratively, a plurality of temperature sensors (not shown) is disposed within a heap in each cell 202 while other temperature sensors disposed outside the cells 202 are used to monitor the outside temperature. Each sensor within a cell 202 is linked to the corresponding blower through a control system and programming of the PLC helps establish ventilation conditions, which are typically based on the external temperature as well as on the temperatures recorded within each cell 202. As a result, aerobic conditions are promoted while excess heat is evacuated to control the temperature within each heap and favour evaporation.

Still referring to FIG. 4, after the organic material has been through the A and B stages of the first composting phase, the partially composted material exiting cells 202 is routed to a primary refining area 204 via a feed hopper 206 and conveyor belts 208. This stage of the composting process aims at extracting the larger sized fraction of the compost material (having a size illustratively superior to 25 mm), which will be routed to the burial facility, and recuperating the fraction having a smaller size (illustratively between 25 mm and 12 mm) mainly composed of bulking agent material. This bulking agent material is immediately recycled and stored in storage area 132 (shown in FIG. 1) for use in the early stages of subsequent composting processes. The separation may be implemented using pneumatic and/or ballistic methods (e.g. a ballistic separator 210) and star screeners 212 may be used to improve efficiency and throughput. In order to obtain a higher separation yield, the material to be screened should be as dry as possible, which is the case of the compost material routed to the primary refining stage. Indeed, the compost material obtained from stages A and B of the first composting phase exhibits a low level of humidity, illustratively less than 45%, thus ensuring a proper separation.

Still referring to FIG. 4, a transition and adjustment phase area 300 is used to control the start of a second phase of organic material composting through re-humidification and forced aeration. The chemical properties of the compost mixture may also be adjusted during this phase. The area 300 comprises a module of two impervious concrete juxtaposed cells 302 illustratively having a nominal capacity of about 180 cubic meters each. At this stage of the composting process, the retention time is relatively short, illustratively of about one week in the cells 302 and the composting reaction is aggressively started. The cells 302 are defined by three reinforced concrete walls and are isolated against thermal losses. Nominal load height is about 3 meters with the organic material being distributed along the cells 302 in peaked piles by a pair of augers as in 304. Similarly to cells 202 in the A and B stages of the first composting phase described herein above, each cell 302 in the transitory and adjustment phase area 300 possesses a concrete floor, in which a strong and rugged network of air ducts (not shown) is embedded to diffuse air under the heaps of organic material in a uniform manner. As before, the ducts also act as drains to allow capture and drainage of wastewater towards the cistern. Similar to cells 202, each cell 302 also a dedicated blower (not shown), which is under control of a PLC or the like, for providing ventilation to the heaps. Temperature sensors are also used to ensure that ventilation conditions are established according to temperature variations inside as well as outside of cells 302, thus favouring aerobic conditions and evaporation.

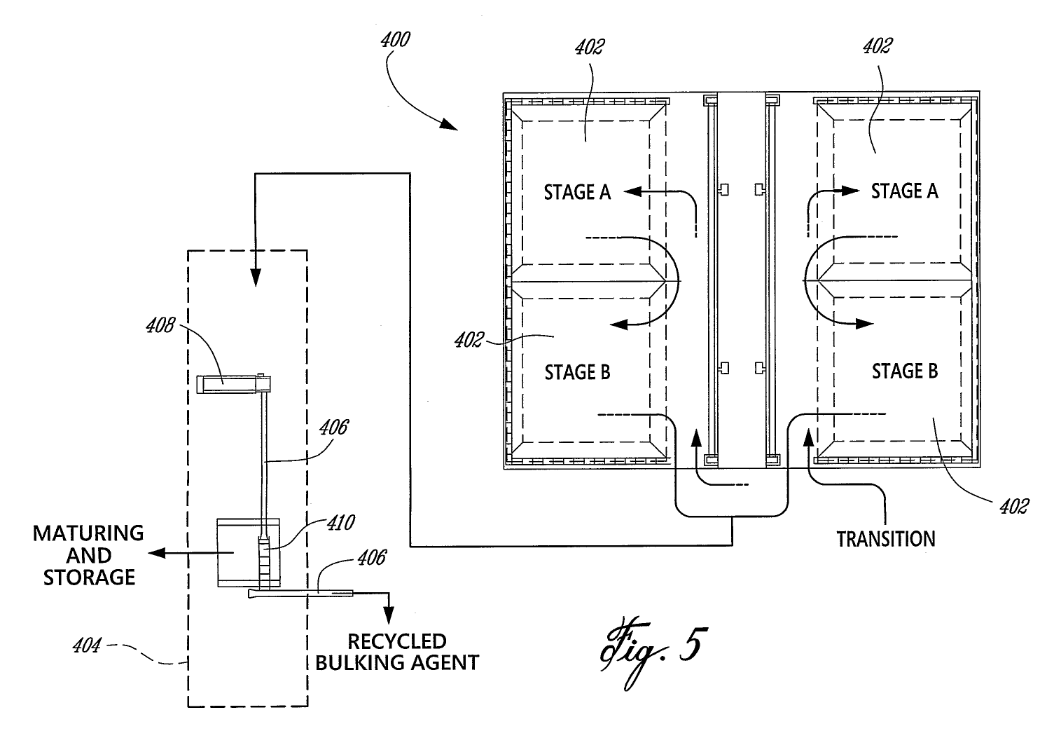

Referring now to FIG. 5, compost leaving the adjustment and transition stage area 300 is routed to the second composting phase area 400 where static piling with forced aeration is implemented to produce high-quality compost. This second composting phase 400 is divided into two modules of two juxtaposed cells 402 illustratively located in two greenhouse buildings. One module thus comprises two heaps, each corresponding to a distinct stage (A or B) of the second composting phase. Each stage A or B illustratively has a retention time of two weeks, with homogenisation, intermingling and re-humidification being required when the compost material is moved from one stage to the next. Since only a small reduction in the volume of the compost material is expected at this stage, all cells 402 have an identical nominal capacity, illustratively about 680 cubic meters. Similar to the first composting phase described herein above, mixing and moving of the organic material is illustratively performed using a loading device, such as a loader on wheels, with the nominal loading height being of about 2.5 meters. Unlike cells 202 however, the cells 402 in the second composting phase area 400 are not defined by concrete walls or insulated against thermal losses. Still, each cell 402 has a network of ducts and air distribution grids embedded in its floor and a dedicated PLC-controlled blower. Temperature sensors are also used to control the temperature within each cell 402.

Still referring to FIG. 5, the compost material is further refined during a secondary refining phase in area 404, which uses equipment (i.e. conveyor belts 406, feed hopper 408) similar to that used in the primary refining area 204. The secondary refining stage 404 aims at recovering the fraction of composted organic material having a pre-determined size, illustratively between 6 and 12 mm. As is the case for partially composted material exiting the first composting phase area 200, this fraction of compost material contains an amount of bulking agent which can be recovered, recycled and stored in storage area 134 (shown in FIG. 1) for later use in the early stages of subsequent composting processes. For the same reasons as the ones given in relation to the discussion of the primary refining stage area 204 herein above, a high separation yield is expected at this point. Indeed, after going through stages A and B of the second composting phase, the compost material reaching the secondary refining area 404 has a low humidity level, illustratively less than 45%, which ensures proper screening. As is the case in the primary refining stage, star screeners 410 are illustratively used to improve efficiency and throughput. To prevent cross-contamination from pathogens, which is higher during the secondary refining stage, equipment (e.g. loading buckets) is not shared between the various stages of the composting process.

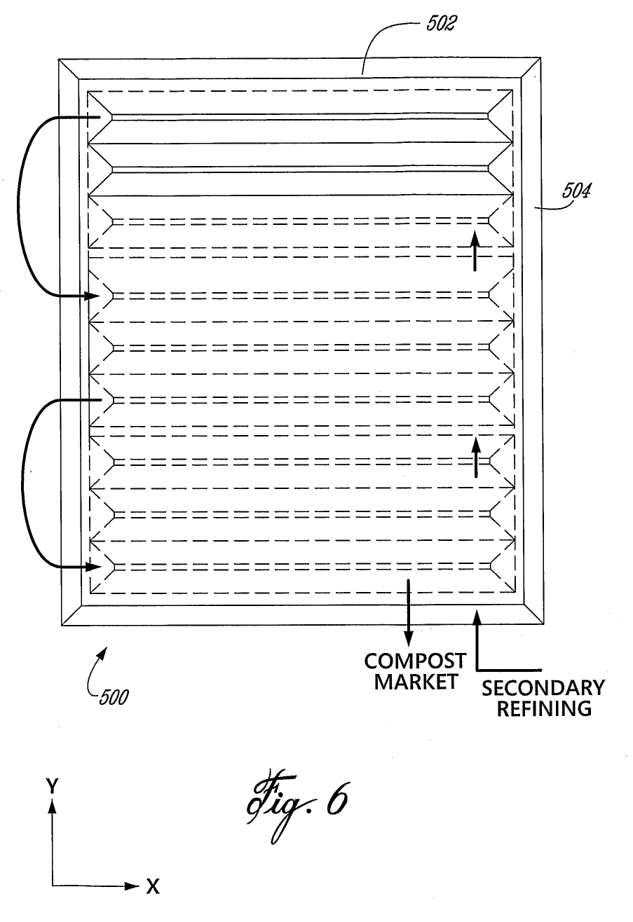

Referring now to FIG. 6, after the second composting phase 400, the fine organic fraction (illustratively having a size inferior to 6 mm) of the compost material is routed to curing and storage area 500 consisting of an open-air curing and storage platform formed by a one-piece compacted concrete slab 502. The slab 502 is typically shaped with a substantially small slope on the two major axes X and Y, illustratively about 2%, to promote bidirectional run-off of liquids. An external peripheral strip 504, illustratively having a width of 3.4 meters, is shaped with a substantially greater counter slope, illustratively 5%. This promotes cleanliness of the procedures while focusing wastewater capture within a narrow peripheral strip located at the edge of the major slope and the counter slope of the slab 502. Manholes (not shown) are also illustratively disposed at the four corners as well as at the centre of the slab 502 for draining wastewater towards a cistern (not shown) via underground pipelines.

It will now be apparent to one of ordinary skill in the art that the curing and storage area 500 may be designed and operated in a variety of fashions. It is desirable however to use a wheel loader for handling the compost material entering the curing phase. This handling involves forming large-sized heaps, which are stirred on a regular basis to promote air circulation during a retention period that may illustratively reach up to nine months. The size, shape and placement of these heaps vary depending on the production requirements as well as the seasons. Trucks, for instance, may use the space between heaps to load the finished compost material, which will be routed to the compost market. The retention period further enables to manage production and retail processes according to the seasons and the market requirements.

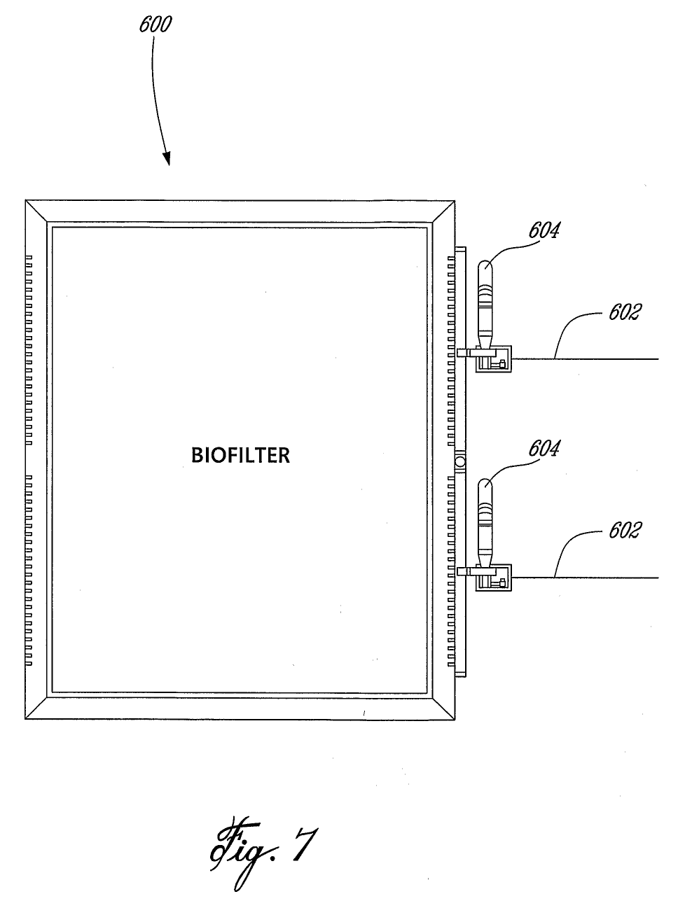

Referring now to FIG. 7, the most intensive composting activity occurs in the first composting phase area 200, resulting in the highest production of noxious gases or fumes in that area of the building. These fumes are extracted using a network of ducts and adjustable vents or inlets arranged above the composting cells 202 and in the circulation area where plant personnel operates. The duct network is illustratively linked via two (2) lines 602 to two (2) blowers 604, which feed the main line of the biofilter 600. The use of two (2) blowers 604 ensures that half of the system remains operational, i.e. with the odour-treatment system functioning and the building kept under negative pressure, in the event of maintenance or equipment failure. The amount of gases or fumes extracted illustratively corresponds to between four (4) and eight (8) air changes per hour. In the transition cell 300, some composting activity still occurs and some fumes are therefore extracted, with the extraction being illustratively equivalent to between four (4) and eight (8) changes per hour. The extraction duct is directly connected to ducts linking the blowers 604 to the biofilter 600. Extraction ducts from the primary refining area 204 and from the wastewater cistern are also illustratively connected to the biofilter 600. Although these ducts have a substantially low rate of flow, they allow a localized ventilation to be maintained in these areas of the plant. The second composting phase area 400 is also illustratively connected to the biofilter 600. However, this is not absolutely necessary as weaker noxious gases or fumes are released at this stage since the compost material has gone through several weeks, illustratively five (5), of intensive aerobic degradation by composting bacteria. Still, for security purposes, the second composting phase area 400 is illustratively also maintained under negative pressure with fumes extracted at a rate of between four (4) and eight (8) changes per hour.

Still referring to FIG. 7, the biofilter 600 gathers noxious emissions from the plant and further cleans contaminated air. It is placed outside of the building where the composting process occurs and is comprised of a rock aggregate, on which screening organic material (e.g. wood chips and other ligneous material) resistant to degradation by micro-organisms is installed. Humid and contaminated air extracted from the odour-generating areas of the main building is distributed through the screening material by a network of pipes embedded in the rock aggregate. Contaminated air remains in contact with the screening material for a short period of time, illustratively 60 seconds for a newly constructed biofilter 600. This allows for bacteria to develop and metabolize by adsorption of the gases, which deposit on the surface of the screening material. This aerobic process has the advantage of producing a minimal proportion of greenhouse effect gases, making the process environmentally friendly.

The temperature of the air incoming into the biofilter 600, and thus that of the screening environment, is illustratively maintained between 40 and 60 degrees Celsius. Humidity of the screening environment is also maintained by water saturation of the contaminated air and/or surface watering of the biofilter 600. In the event of heavy rain, excess water is collected by a watertight membrane installed under the rock aggregate of the biofilter 600 and water collected is channelled towards the wastewater cistern. The performance of the biofilter 600, i.e. proper elimination of odours, is influenced by the properties of the contaminated air and of the screening organic material as well as the interaction time between the two. Properties of the contaminated air include its humidity level, its flow rate and the ratio of volatile organic compound (i.e. odours) it contains for example. Properties of the screening organic material depend on the selection of the organic components and include its porosity, its water retention capacity and its ability to absorb volatile organic compounds contained in the contaminated air. It is desirable to choose organic materials such as cedar chips, sphagnum moss, peat wood, very mature compost, or other ligneous materials. It is also desirable for the mechanical structure of the mix of organic material to be composed of material with varied granularity. This will ensure a long-term resistance to separation of the particulates as well as to gradual subsidence of the heap. Illustratively, the porosity of the initial screening mix is selected to be of about 60%.

As mentioned herein above, an outside cistern (not shown) fabricated from impervious concrete may illustratively be used to collect wastewater generated by the overall composting process. Water from the site may be drained using gravity as well as pumps or other means for displacing liquids, which may be used in some cases to ensure proper fluid transport. Water collected in the cistern is further re-used for humidifying the compost at different stages of the process.

Although the present invention has been described hereinabove by way of specific embodiments thereof, it can be modified, without departing from the spirit and nature of the subject invention as defined in the appended claims.

1. A system for treating a waste material comprising a predominant amount by weight of a compostable organic material, the system comprising:
a mixer for combining a bulking agent with the waste material;
a first sorting device for removing predominantly inorganic material having a dimension exceeding a predetermined amount from said bulked waste material;
a first phase compositing area located in an enclosed area for receiving said sorted waste material on a floor thereof, said first phase area further comprising:
a first network of drains and a first network of air ducts imbedded in said first phase compositing area floor, said first network of drains leading to a cistern and said first network of air ducts in operative engagement with a blower for blowing air out of said first network of air ducts;
a means for increasing a humidity of said sorted waste material;
a means for inoculating said sorted waste material with a composting bacteria;
a first network of vents for collecting noxious gases emitted by said composting bacteria when compositing said sorted waste material and transmitting said emitted gases to a bio-filter;
a second sorting device for removing residual inorganic material and said bulking agent from said partially composted sorted waste material;
a transition area for receiving partially composted sorted waste material from said first phase compositing area on a floor thereof;
a second phase compositing area for receiving the compostable organic material from said transition area on a floor thereof, said second phase area comprising:
a second network of drains and a second network of air ducts imbedded in said second phase compositing area floor, said second network of drains leading to said cistern and said second network of air ducts in operative engagement with said blower for blowing air through said second network of air ducts;
a means for increasing a humidity of the compostable organic material; and
a means for re-inoculating the compostable organic material with said composting bacteria; and
a curing phase compositing area for storing composted organic material.

2. (canceled)

3. (canceled)

4. (canceled)

5. (canceled)

6. The system of claim 1, wherein said transition area is located in an enclosed area and further comprises:
a third network of drains and a third network of air ducts imbedded in said transition area floor, said third network of drains leading to a cistern and said third network of air ducts in operative engagement with a blower for blowing air out of said third network of air ducts;
a means for increasing a humidity of the compostable organic material;
a means for re-inoculating the compostable organic material with a composting bacteria; and
a third network of vents for collecting noxious gases emitted by said composting bacteria when compositing the compostable organic material and transmitting said emitted gases to said bio-filter.

7. The system of claim 1, wherein said transition area further comprises a means for adjusting a chemical composition of the compostable organic material following removal of said residual inorganic material and said bulking agent from said sorted waste material.

8. The system of claim 1, wherein said second phase compositing area is located in an enclosed area and further wherein said second phase compositing area comprises a third network of vents for collecting noxious gases emitted by said composting bacteria when compositing the compostable organic material and transmitting said emitted gases to said bio-filter.

9. (canceled)

10. (canceled)

11. (canceled)

12. (canceled)

13. (canceled)

14. (canceled)

15. The system of claim 1, wherein said inoculating means comprises a plurality of sprinklers arranged above said floor, said sprinklers in operative engagement with a source of water under pressure containing said compositing bacteria.

16. The system of claim 15, wherein water retained in said cistern contains said compositing bacteria and further wherein said source of water under pressure comprises a pump in operative engagement with said cistern.

17. A method for treating a waste material comprising a predominant amount by weight of a compostable organic material, the method comprising:
during a conditioning phase:
adding a bulking agent to the waste material;
pretreating said bulked waste material to remove predominantly inorganic material having at least one dimension of greater than a predetermined amount;
during an initial composting phase:
piling said pretreated waste material into a first heap;
promoting drying and compositing of the pretreated waste material in said heap by permeating air into said heap;
collecting gases generated by a composting bacteria during compositing said pretreated waste material; and
treating said collected gases in a bio-filter;
during a subsequent composting phase:
removing residual inorganic material and a majority of said bulking agent from said dried and partially composted waste material to yield partially composted organic material;
humidifying said partially composted organic material by adding water; and
piling said partially composted organic material into a second heap; and
promoting drying and compositing of said partially composted organic material by permeating air into said heap;
during a curing phase:
removing residual bulking agent from said dried composted organic material; and
storing said dried composted organic material.

18. The method of claim 17, further comprising the act, during said initial composting phase, of inoculating said waste material with said bacteria.

19. (canceled)

20. The method of claim 17, wherein during said initial composting phase following said drying promoting and compositing act, said waste material is re-humidified and said drying promoting and compositing act repeated.

21. The method of claim 17, wherein during said subsequent composting phase following said drying promoting and compositing act, said partially composted organic material is re-humidified and said drying promoting and compositing act repeated.

22. (canceled)

23. (canceled)

24. (canceled)

25. (canceled)

26. The method of claim 17, wherein said permeating air act is cyclic.

27. (canceled)

28. (canceled)

29. The method of claim 17, wherein during said initial composting phase, said promoting act further comprises adding additional moisture to said pretreated waste after a predetermined period of time.

30. (canceled)

31. (canceled)

32. (canceled)

33. (canceled)

34. The method of claim 17, wherein said bio-filter comprises a support material selected from the group consisting of tree bark, cedar chips, sphagnum moss, peat wood and mature compost inoculated with a compositing bacteria and further wherein said collected gases treating act comprises forcing said collected gases through said inoculated support material.

35. The method of claim 17, wherein a predominant amount of the waste material is retained in a plurality of plastic bags and further comprising the act of tearing at least one hole in each of said plastic bags prior to said bulking agent adding act.

36. The method of claim 17, wherein said inoculating act comprises spraying said heap with water containing said composting bacteria.

37. (canceled)

38. (canceled)

39. (canceled)

40. (canceled)

41. The method of claim 17, wherein said residual bulking agent removed during said subsequent composting phase is recycled and reused during said conditioning phase.

42. The method of claim 17, wherein said bulking agent removed during said curing phase is recycled and reused during said conditioning phase.

43. A method for compositing waste material comprising a predominant amount of compostable organic material, the method comprising:
homogenously mixing said waste material with a bulking agent, wherein a predominant amount of said bulking agent is recycled bulking agent;
spraying said waste material and bulking agent mix with a water containing a composting bacteria;
promoting drying and partial compositing of the composting waste material by permeating air through the waste material and bulking agent mix;
separating said bulking agent from said partially composted waste material to yield partially composted organic material and said recycled bulking agent;
spraying said partially composted waste material with a water containing a composting bacteria; and
promoting drying and compositing of the compostable organic material by permeating air through said waste material and said bulking agent.

44. (canceled)

45. A method for mixing waste material comprising a predominant amount of compostable organic material with a bulking agent, the method comprising:
providing a mixer comprising a mixing tub comprising an inverted frusto conical shape and a closed lower end and a vertical auger mounted for rotation in said lower end, said auger comprised of at least one exposed helical flighting extending from a frusto-conical hub;
placing the waste material in said mixing tub;
mixing the waste material by rotating said auger in a direction of said helical flighting at a first speed;
adding the bulking agent to the mixed waste material; and
mixing the bulking agent and the waste material by rotating said auger in a direction of said helical flighting at a second speed less than said first speed.

46. The method of claim 45, wherein said waste material is Source Separated Organic (SSO) material, a predominant amount of said SSO material retained in plastic bags, said mixer further comprises a plurality of serrated blades mounted along an outer edge of said flighting and wherein said waste material mixing act comprises tearing open said bags using said blades.

47. (canceled)

48. (canceled)
